(19) United States
(12) Patent Application Publication
(10) Pub. No.: US 2026/0137394 A1
(43) Pub. Date: May 21, 2026

(54) SYSTEM AND METHOD FOR DETECTING BLADE DEFLECTION DURING ORTHOPAEDIC SURGICAL PROCEDURES

(71) Applicant: DEPUY IRELAND UNLIMITED COMPANY, RINGASKIDDY (IE)

(72) Inventor: Narendra D. Vaidya, Hudson, NH (US)

(21) Appl. No.: 18/949,122

(22) Filed: Nov. 15, 2024

(51) Int. Cl. A61B 17/14

(52) U.S. Cl. CPC A61B 17/144

(57) ABSTRACT

An orthopaedic surgical procedure includes monitoring output from a number of onboard sensors of a bone saw to detect the presence of blade deflection during advancement of the saw’s oscillating blade into the bone of a patient. Operation of the bone saw is adjusted based on the amount of detected blade deflection. A robot-assisted orthopaedic surgical system is also disclosed.

### TECHNICAL FIELD

The present disclosure relates generally to orthopaedic surgical procedures and, more particularly, to systems and methods for detecting blade deflection during orthopaedic surgical procedures.

### BACKGROUND

Joint arthroplasty is a well-known surgical procedure by which a diseased and/or damaged natural joint is replaced by a prosthetic joint, which may include one or more orthopaedic implants. To facilitate the replacement of the natural joint with the prosthetic joint, orthopaedic surgeons may use a variety of orthopaedic surgical instruments such as, for example, surgical saws, cutting guides, reamers, broaches, drill guides, drills, positioners, insertion tools and/or other surgical instruments. A surgeon may use manual instruments such as cutting blocks or other cutting guides to perform the various resections in an orthopaedic procedure. Alternatively, or in addition, a surgeon may use a computer-assisted surgical navigation system, such as a robotic-assisted surgical system, to perform the various resections in an orthopaedic procedure.

A robotic-assisted surgical system may be used to perform a total knee arthroplasty (TKA) or unicompartmental knee arthroplasty (UKA) surgical procedure. Typical robotic-assisted surgical systems use a bone saw that is constrained by the system to perform resections along preplanned resection planes.

### SUMMARY

According to one aspect, a method of operating a robotic-assisted orthopaedic surgical system includes controlling, by a computer system, a bone saw to oscillate a bone saw blade and operating, by the computer system, a sensor assembly positioned in the bone saw to determine an amount of blade deflection in the bone saw blade. Operation of the bone saw is adjusted by the computer system based on the determined amount of blade deflection of the bone saw blade.

Speed of oscillation of the bone saw blade may be adjusted by the computer system based on the determined amount of blade deflection of the bone saw blade. In an embodiment, speed of oscillation of the bone saw blade is slowed by the computer system based on the determined amount of blade deflection of the bone saw blade.

In an embodiment, a cutting angle of the bone saw blade is adjusted by the computer system based on the determined amount of blade deflection of the bone saw blade.

The sensor assembly positioned in the bone saw may be operated by the computer system to determine an amount of in-plane blade deflection of the bone saw blade.

The sensor assembly positioned in the bone saw may be operated by the computer system to determine an amount of out-of-plane blade deflection of the bone saw blade.

In an embodiment, the bone saw is operated during advancement thereof into a bone of a patient.

The sensor assembly positioned in the bone saw may be operated by the computer system to determine an amount of blade deflection in a linear array pattern on the bone saw blade.

The sensor assembly positioned in the bone saw may be operated by the computer system to determine an amount of blade deflection in a grid array pattern on the bone saw blade.

A monitor device of the orthopaedic surgical system may be operated by the computer system to generate an audible and/or visual feedback message based on the determined amount of blade deflection of the bone saw blade.

According to another aspect, an orthopaedic surgical system includes a robotic-assisted bone saw having a sensor assembly housed therein and a computer system electrically coupled to the bone saw. The computer system is configured to control the bone saw to oscillate a bone saw blade, operate the sensor assembly to determine an amount of blade deflection in the bone saw blade, and adjust operation of the bone saw based on the determined amount of blade deflection of the bone saw blade.

The computer system may be further configured to adjust speed of oscillation of the bone saw blade based on the determined amount of blade deflection of the bone saw blade.

In an embodiment, the computer system is further configured to slow speed of oscillation of the bone saw blade based on the determined amount of blade deflection of the bone saw blade.

The computer system may be further configured to adjust a cutting angle of the bone saw blade based on the determined amount of blade deflection of the bone saw blade.

In an embodiment, the computer system is further configured to operate the sensor assembly positioned in the bone saw to determine an amount of in-plane blade deflection of the bone saw blade.

In one embodiment, the computer system is further configured to operate the sensor assembly positioned in the bone saw to determine an amount of out-of-plane blade deflection of the bone saw blade.

The computer system may be further configured to operate the bone saw to oscillate the bone saw blade during advancement of the bone saw blade into a bone of a patient.

In an embodiment, the computer system is further configured to operate the sensor assembly positioned in the bone saw to determine an amount of blade deflection in a linear array pattern on the bone saw blade.

In one embodiment, the computer system is further configured to operate the sensor assembly positioned in the bone saw to determine an amount of blade deflection in a grid array pattern on the bone saw blade.

The orthopaedic surgical system may also include a monitor device. The computer system may be further configured to operate the monitor device to generate an audible and/or visual feedback message based on the determined amount of blade deflection of the bone saw blade.

### BRIEF DESCRIPTION OF THE DRAWINGS

The concepts described herein are illustrated by way of example and not by way of limitation in the accompanying figures. For simplicity and clarity of illustration, elements illustrated in the figures are not necessarily drawn to scale. Where considered appropriate, reference labels have been repeated among the figures to indicate corresponding or analogous elements. The detailed description particularly refers to the accompanying figures in which:

FIG. 1 is a schematic diagram of a system for planning and assisting an orthopaedic surgical procedure;

FIGS. 2 and 3 are perspective views of an oscillating bone saw of the robotic surgical device of the system of FIG. 1;

FIG. 4 is a schematic illustration demonstrating in-plane blade deflection;

FIG. 5 is a schematic illustration demonstrating out-of-plane blade deflection; and

FIG. 6 is a simplified flow diagram of a method for an orthopaedic surgical procedure including automated detecting of blade deflection that may be executed by the surgical planning and assistance device of FIG. 1.

### DETAILED DESCRIPTION OF THE DRAWINGS

While the concepts of the present disclosure are susceptible to various modifications and alternative forms, specific embodiments thereof have been shown by way of example in the drawings and will be described herein in detail. It should be understood, however, that there is no intent to limit the concepts of the present disclosure to the particular forms disclosed, but on the contrary, the intention is to cover all modifications, equivalents, and alternatives consistent with the present disclosure and the appended claims.

Terms representing anatomical references, such as anterior, posterior, medial, lateral, superior, inferior, proximal, distal, etcetera, may be used throughout the specification in reference to the orthopaedic implants or prostheses and surgical instruments described herein as well as in reference to the patient’s natural anatomy. Such terms have well-understood meanings in both the study of anatomy and the field of orthopaedics. Use of such anatomical reference terms in the written description and claims is intended to be consistent with their well-understood meanings unless noted otherwise.

References in the specification to “one embodiment,” “an embodiment,” “an illustrative embodiment,” etc., indicate that the embodiment described may include a particular feature, structure, or characteristic, but every embodiment may or may not necessarily include that particular feature, structure, or characteristic. Moreover, such phrases are not necessarily referring to the same embodiment. Further, when a particular feature, structure, or characteristic is described in connection with an embodiment, it is submitted that it is within the knowledge of one skilled in the art to effect such feature, structure, or characteristic in connection with other embodiments whether or not explicitly described. Additionally, it should be appreciated that items included in a list in the form of “at least one A, B, and C” can mean (A); (B); (C); (A and B); (A and C); (B and C); or (A, B, and C). Similarly, items listed in the form of “at least one of A, B, or C” can mean (A); (B); (C); (A and B); (A and C); (B and C); or (A, B, and C).

The disclosed embodiments may be implemented, in some cases, in hardware, firmware, software, or any combination thereof. The disclosed embodiments may also be implemented as instructions carried by or stored on a transitory or non-transitory machine-readable (e.g., computer-readable) storage medium, which may be read and executed by one or more processors. A machine-readable storage medium may be embodied as any storage device, mechanism, or other physical structure for storing or transmitting information in a form readable by a machine (e.g., a volatile or non-volatile memory, a media disc, or other media device).

In the drawings, some structural or method features may be shown in specific arrangements and/or orderings. However, it should be appreciated that such specific arrangements and/or orderings may not be required. Rather, in some embodiments, such features may be arranged in a different manner and/or order than shown in the illustrative figures. Additionally, the inclusion of a structural or method feature in a particular figure is not meant to imply that such feature is required in all embodiments and, in some embodiments, may not be included or may be combined with other features.

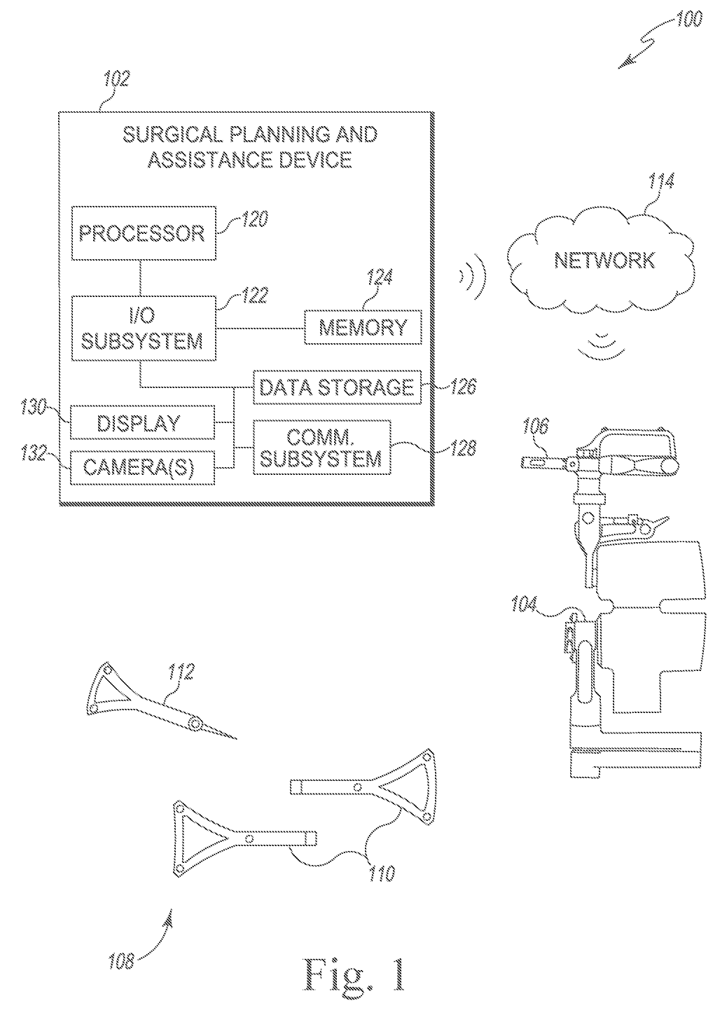

Referring now to FIG. 1, a surgical system 100 is used during an orthopaedic surgical procedure, which is illustratively a joint replacement procedure such as a total knee arthroplasty (TKA) procedure. During that procedure, an orthopaedic surgeon performs registration of the patient’s anatomy with the system 100. The surgeon or other user uses a surgical planning and assistance device 102 to automatically create a surgical plan based on joint space values provided by the surgeon. For example, the surgeon provides target extension space and flexion space values, which represent desired ligament balance for the knee joint after the surgical procedure, and the device 102 determines appropriate resection planes to achieve the target joint space values. A robotic surgical device 104 may be controlled based on the surgical plan during operation of the surgical procedure, for example by robotically constraining a surgical bone saw 106 to one or more resection planes defined by the surgical plan.

As shown in FIG. 1, the system 100 includes the surgical planning and assistance device 102 and the robotic surgical device 104 as well as multiple registration targets 108. The surgical planning and assistance device 102 may be embodied as any type of computer system capable of performing the functions described herein. For example, the surgical planning and assistance device 102 may be embodied as, without limitation, a workstation, a desktop computer, a laptop computer, a special-purpose compute device, a server, a rack-mounted server, a blade server, a network appliance, a web appliance, a tablet computer, a smartphone, a consumer electronic device, a distributed computing system, a multiprocessor system, and/or any other computing device capable of performing the functions described herein. Additionally, although the surgical planning and assistance device 102 is illustrated in FIG. 1 as embodied as a single computer, it should be appreciated that the surgical planning and assistance device 102 may be embodied as multiple devices cooperating together to facilitate the functionality described below. For example, in some embodiments the system 100 may include a base station and a satellite station or other combination of computing devices. Additionally or alternatively, in some embodiments, the surgical planning and assistance device 102 may be embodied as a “virtual server” formed from multiple computer systems distributed across a network and operating in a public or private cloud.

As shown in FIG. 1, the illustrative surgical planning and assistance device 102 includes a processor 120, an I/O subsystem 122, memory 124, a data storage device 126, and a communication subsystem 128. Of course, the surgical planning and assistance device 102 may include other or additional components, such as those commonly found in a computer (e.g., various input/output devices), in other embodiments. Additionally, in some embodiments, one or more of the illustrative components may be incorporated in, or otherwise form a portion of, another component. For example, the memory 124, or portions thereof, may be incorporated in the processor 120 in some embodiments.

The processor 120 may be embodied as any type of processor or controller capable of performing the functions described herein. For example, the processor may be embodied as a single or multi-core processor(s), digital signal processor, microcontroller, or other processor or processing/controlling circuit. Similarly, the memory 124 may be embodied as any type of volatile or non-volatile memory or data storage capable of performing the functions described herein. In operation, the memory 124 may store various data and software used during operation of the surgical planning and assistance device 102 such as operating systems, applications, programs, libraries, and drivers. The memory 124 is communicatively coupled to the processor 120 via the I/O subsystem 122, which may be embodied as circuitry and/or components to facilitate input/output operations with the processor 120, the memory 124, and other components of the surgical planning and assistance device 102. For example, the I/O subsystem 122 may be embodied as, or otherwise include, memory controller hubs, input/output control hubs, firmware devices, communication links (i.e., point-to-point links, bus links, wires, cables, light guides, printed circuit board traces, etc.) and/or other components and subsystems to facilitate the input/output operations. In some embodiments, the I/O subsystem 122 may form a portion of a system-on-a-chip (SoC) and be incorporated, along with the processor 120, the memory 124, and other components of the surgical planning and assistance device 102, on a single integrated circuit chip.

The data storage device 126 may be embodied as any type of device or devices configured for short-term or long-term storage of data such as, for example, memory devices and circuits, memory cards, hard disk drives, solid-state drives, or other data storage devices. The communication subsystem 128 of the surgical planning and assistance device 102 may be embodied as any communication circuit, device, or collection thereof, capable of enabling communications between the surgical planning and assistance device 102 and remote devices. The communication subsystem 128 may be configured to use any one or more communication technology (e.g., wireless or wired communications) and associated protocols (e.g., Ethernet, Bluetooth®, Wi-Fi®, WiMAX, etc.) to effect such communication.

As shown in FIG. 1, the surgical planning and assistance device 102 includes a display 130. The display 130 may be embodied as any type of display capable of displaying digital images or other information, such as a liquid crystal display (LCD), a light emitting diode (LED), a plasma display, a cathode ray tube (CRT), or other type of display device. In some embodiments, the display 130 may be coupled to a touch screen to allow user interaction with the surgical planning and assistance device 102. The display 130 may also include one or more speakers and is thus operable to generate audible messages in addition to visual messages.

The surgical planning and assistance device 102 further includes one or more cameras 132. Each of the cameras 132 may be embodied as a digital camera or other digital imaging device coupled to the surgical planning and assistance device 102. Each camera 132 includes an electronic image sensor, such as an active-pixel sensor (APS), e.g., a complementary metal-oxide-semiconductor (CMOS) sensor, or a charge-coupled device (CCD). In the illustrative embodiment, multiple cameras 132 are arranged in an array and are thus capable of determining distance to objects imaged by the cameras 132.

The robotic surgical device 104 may be embodied as any type of robot capable of performing the functions described herein. Illustratively, the robotic surgical device 104 is embodied as a robotic arm that may be attached to a surgical table or otherwise positioned near a patient during the orthopaedic surgical procedure. The robotic surgical device 104 includes a surgical tool 106, illustratively embodied as a surgical bone saw 106. In use, the robotic surgical device 104 supports the surgical bone saw 106 and may constrain movement of the surgical bone saw 106 within a resection plane specified in a surgical plan. The surgeon may activate the surgical saw 106 and perform the resection with the surgical saw 106 while the robotic surgical device 104 constrains movement of the surgical saw 106 to the resection plane. The robotic surgical device 104 may illustratively be embodied as a VELYS™ Robotic-Assisted Solution, commercially available from DePuy Synthes Products, Inc. of Warsaw, Indiana.

The surgical planning and assistance device 102 and the robotic surgical device 104 may be configured to transmit and receive data with each other and/or other devices of the system 100 over a network 114. The network 114 may be embodied as any number of various wired and/or wireless networks. For example, the network 114 may be embodied as, or otherwise include, a wired or wireless local area network (LAN), a wired or wireless wide area network (WAN), a cellular network, and/or a publicly-accessible, global network such as the Internet. As such, the network 114 include any number of additional devices, such as additional computers, routers, stations, and switches, to facilitate communications among the devices of the system 100.

As shown in FIG. 1, the system 100 further includes a number of registration tools 108. In use, the surgical planning and assistance device 102 may track the location of the registration tools 108 in space using the array of cameras 132. For example, each registration tool 108 may include a number of hydrophobic optical reflectors arranged in a predetermined pattern visible to the cameras 132. Illustratively, the registration tools 108 include a plurality of arrays 110 configured to each be secured to one of the patient’s bones, to the robotic surgical device 104, or to the surgical tool 106. Illustratively, the registration tools 108 also include a pointer 112 configured to be temporarily positioned by a surgeon relative to anatomical landmarks of the patient (e.g., with an end of the pointer 112 in contact those anatomical landmarks) while the pointer 112 is observed by the cameras 132. As such, the registration tools 108 may be used for registration and tracking of the patient’s bony anatomy during the orthopaedic surgical procedure. Although illustrated as including registration tools 108 suitable for optical tracking with the cameras 132, it should be understood that in some embodiments, the system 100 may perform electromagnetic tracking or other position tracking technology for tracking the registration tools 108.

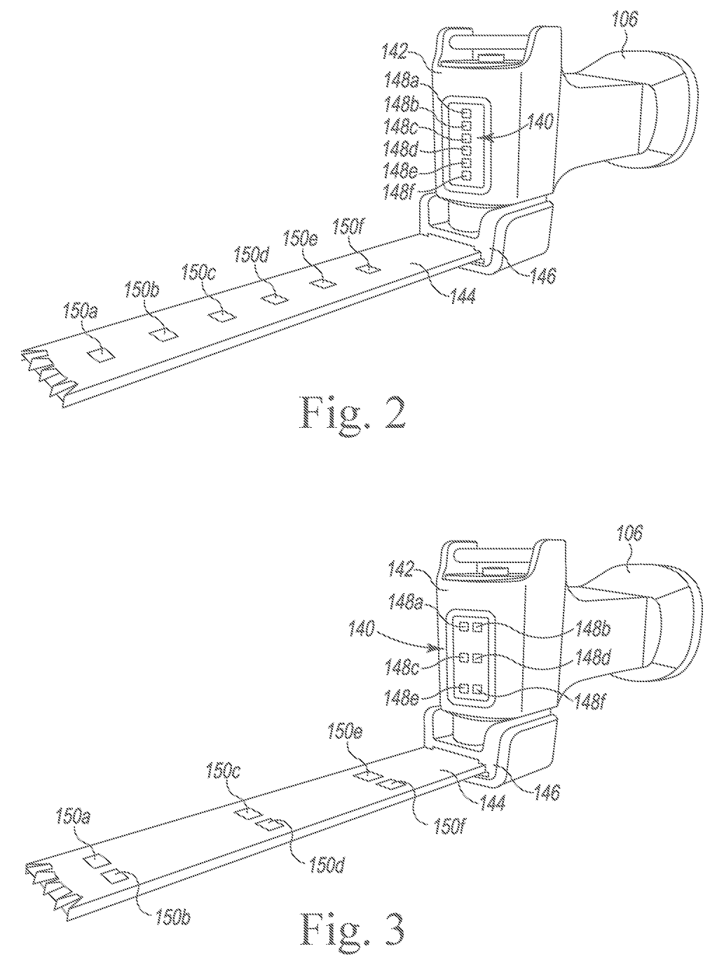

Referring now to FIGS. 2 and 3, the surgical bone saw 106 has an onboard sensor assembly 140 housed in the head 142 of the saw 106. As will be discussed below in more detail, the sensor assembly 140 is operable to detect the presence and magnitude of blade deflection in the oscillating saw blade 144 secured in the saw’s blade clamp 146 as the blade 144 is advanced into the bone of a patient. The sensor assembly 140 includes a number of non-contact sensors 148a, 148b, 148c, 148d, 148e, 148f that are operable to detect multiple target locations 150a, 150b, 150c, 150d, 150e, 150f, respectively, on the saw blade 144 to measure deflection in the blade 144. The individual sensors 148 of the sensor assembly 140 may be embodied as any of numerous types of distance sensors such as time-of-flight (TOF) sensors, light detection and ranging (LiDAR) sensors, laser sensors, ultrasonic sensors, and other commercially-available distance sensors. The individual sensors 148 of the assembly measure the distance to the corresponding target location 150 on the saw blade 144 during movement (i.e., oscillation) of the blade 144 and compare the measured distance to a known expected distance. In such a way, blade deflection can be determined if the measured distance from one or a combination of sensors 148 varies outside of an accepted range (e.g., +/- 2mm).

The individual sensors 148 and the associated target locations 150 may be provided in any number and in any suitable arrangement or pattern to fit the needs of a given design. In the exemplary embodiment described herein, the sensor assembly 140 includes six sensors 148 which are individually operable to measure the distance to six corresponding target areas 150 on the bone saw blade 144. As such, the sensors 148 are spread out along the length of the saw blade 144 thereby allowing blade deflection to be detected even as the blade 144 is advanced further and further into the bone. In other words, as the blade 144 is advanced deeper into the patient’s bone, the sensor assembly 140 may no longer be able to target the target areas near the tip of the blade 144 (e.g., target areas 150a, 150b) since such target areas are masked by the bone. However, because the remaining target areas (e.g., 150c, 150d, 150f, 150e) are spaced out along the remaining length of the blade 144 and are thus not yet advanced into the bone, the sensor assembly 140 may continue to monitor the blade 144 for deflection. As also shown in FIGS. 2 and 3, in the exemplary embodiments described herein, the individual sensors 148 and the associated target locations 150 may be arranged in a linear array pattern (FIG. 2) or a grid array pattern (FIG. 3). In the case of a linear array pattern, a relatively large number of targets along the length of the blade 144 may be monitored. In the case of a grid array pattern, it is possible to monitor deflection on each individual side of the blade 144. In other words, by separately targeting both sides of the blade, it may be determined if the blade 144 is deflecting more on one side versus the other side – e.g., the blade 144 may deflect more as it oscillates to the left versus when it oscillates to the right or vice versa.

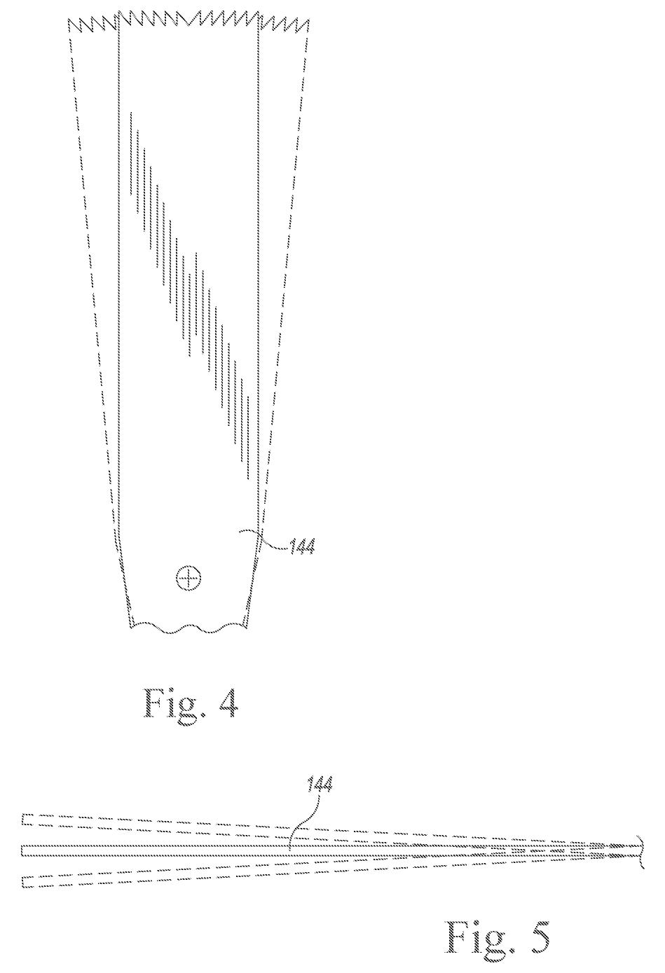

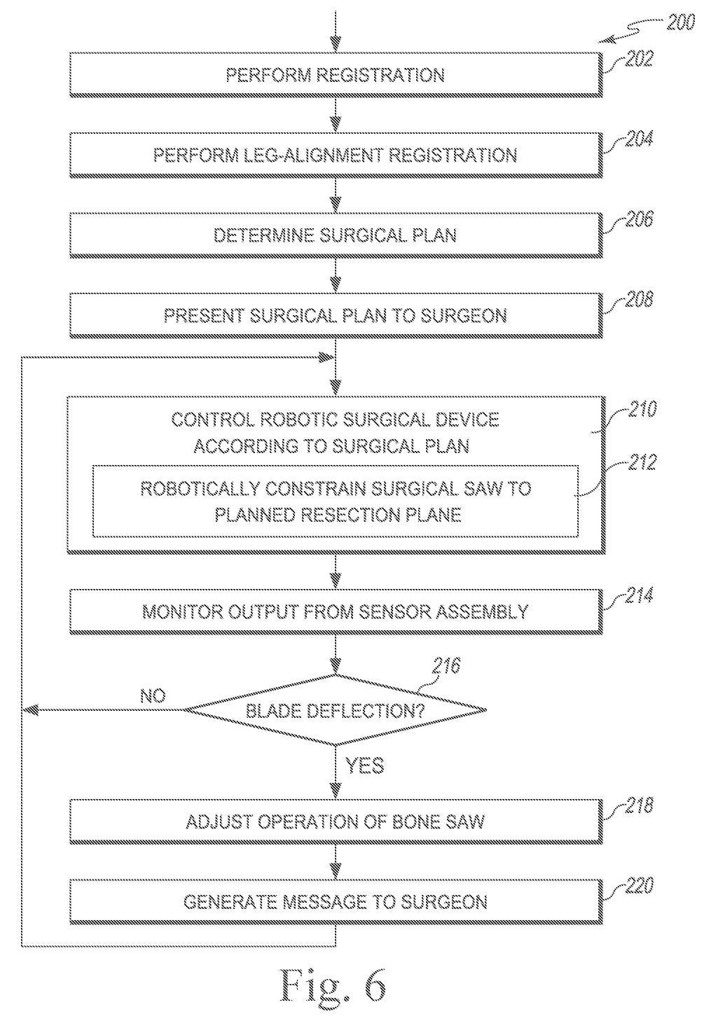

In addition to other arrangements, use of either pattern allows the sensor assembly 140 to detect the presence of either in-plane or out-of-plane blade deflection. Specifically, as shown in FIG. 4, in-plane deflection occurs when the blade 144 deflects to the left or to the right or both (i.e., X-axis) within the cutting plane during oscillation of the blade 144. The sensor assembly 140 is operable to detect such in-plane deflection by comparing the detected position of the blade 144 relative to predetermined side-to-side boundaries. Similarly, as shown in FIG. 5, out-of-plane deflection occurs when the blade 144 deflects up or down or both (i.e., Z-axis) outside of the cutting plane during oscillation of the blade 144. The sensor assembly 140 is operable to detect such out-of-plane deflection by comparing the detected position of the blade 140 relative to predetermined top/upper and bottom/lower boundaries.

During operation of the bone saw 106 the sensor assembly 140 measures the distance to the various target areas 150 on the saw blade 144. The output from the individual sensors 148 of the sensor assembly 140 is monitored by the surgical planning and assistance device 102 to detect the presence of either in-plane or out-of-plane blade deflection by comparing the measured distances to known and/or expected distances. If the surgical planning and assistance device 102 determines an amount of either in-plane or out-of-plane blade deflection is present, the device 102 adjusts operation of the bone saw 106 based on the amount blade deflection present. Based on the amount and type (e.g., in-plane versus out-of-plane) of blade deflection, the surgical planning and assistance device 102 may adjust the oscillation speed of the saw blade 144 by changing the speed of the saw’s motor. In many such cases, the saw’s motor is slowed to slow the speed of the saw blade 144. In some cases, such as when out-of-plan blade deflection is detected, the surgical planning and assistance device 102 may operate the robotic surgical device 104 to adjust the cut angle of the bone saw 106 to accommodate for the detected amount of blade deflection. In some cases, in response to detected blade deflection, the surgical planning and assistance device 102 may operate the robotic surgical device 104 to slow the forward advancement of the bone saw 106 by the surgeon by providing resistance via the device’s robotic arm thereby reducing bending (and hence deflection) of the blade. In certain cases, the device 102 may cease operation of the bone saw 106.

The surgical planning and assistance device 102 may execute other responses if it determines blade deflection is present during operation of the bone saw 106. For example, the surgical planning and assistance device 102 may operate the display 130 to generate a visual and/or audible feedback message to the surgeon that informs the surgeon or other user of the presence and magnitude of blade deflection. The surgical planning and assistance device 102 may also update the calibration files associated with the bone saw 106. In particular, each bone saw 106 is calibrated at regular intervals during its operational life. Amongst other things, the calibration process takes into account any operational characteristics of the saw related to blade deflection. The surgical planning and assistance device 102 may utilize feedback from the sensor assembly 140 to dynamically adjust the calibration settings of a given bone saw 106.

Referring now to FIG. 6, in use, the surgical planning and assistance device 102 may perform a method 200 for an orthopaedic surgical procedure with automated surgical planning. The method 200 begins with block 202, in which the device 102 may perform bony registration of the patient’s bony anatomy. To perform the bony registration, the surgeon may attach a bone array 110 to each of the patient’s tibia and femur. The surgeon may use the pointer 112 to touch various landmarks on the patient’s bony anatomy. During the registration, the device 102 uses the cameras 132 to track the position of the bone arrays 110 and the pointer 112 and thus registers the position of each landmark of the patient’s bony anatomy. As part of bony registration, the device 102 may receive a cartilage loss estimation from the surgeon or other user. The surgeon or another user may input such cartilage loss estimates to the device 102, for example using the touch screen 130 or other input device.

In block 204, the device 102 performs leg-alignment registration to assess the balance of the patient’s knee joint throughout a range of motion. To perform the leg-alignment registration, the surgeon may articulate the patient’s knee joint through the range of motion while the device 102 uses the cameras 132 to track the position of the bone arrays 110 and thus registers the relative positions of the patient’s femur and tibia at multiple points in the range of motion. The surgeon may apply stress on the knee joint as it is moved through the range of motion in order to generate gaps in the joint.

In block 206, the device 102 determines a surgical plan based on target values provided by the surgeon. The surgical plan includes a computed, planned value for each surgical parameter associated with the orthopaedic surgical procedure. In some embodiments, the device 102 may optimize the surgical plan based on preferred resection priority and boundaries provided by the surgeon.

After generating the surgical plan, the device 102 presents the surgical plan to the surgeon or other user in block 208. The device 102 may use any input/output device or output modality to present the surgical plan. In some embodiments, the device 102 may display numerical dimensions for resection heights, angles, position shifts, or other parameters of the surgical plan using the display 130. In some embodiments, the device 102 may graphically display the dimensions of the surgical plan using the display 130. For example, the device 102 may graphically render three-dimensional models of the patient’s bony anatomy along with the prosthetic components that are positioned relative to the bony anatomy according to the surgical plan.

In block 210, the device 102 controls the robotic surgical device 104 according to the surgical plan to assist the surgeon in performing the orthopaedic surgical procedure. The device 102 may transmit the surgical plan to the robotic surgical device 104 or otherwise cause the robotic surgical device 104 to operate according to the surgical plan. Illustratively, in block 212, the robotic surgical device 104 robotically constrains the surgical saw 106 to a number of planned resection planes according to the surgical plan. To do so, the robotic surgical device 104 locates each of the planned resection planes relative to the patient’s anatomy by tracking the bone array 110 using the cameras 132 of the device 102, similar to the bony registration process described above. Under such robotic constraint, the surgical bone saw 106 is operated to oscillate the bone saw blade 144 along the planned resection planes so as to resect the patient’s femur and tibia.

In block 214, the device 102 monitors output from the sensor assembly 140 onboard the bone saw 106 as the saw is used to resect the patient’s femur and tibia along the planned resection planes. Specifically, continuous output from each of the sensors 148a, 148b, 148c, 148d, 148e, 148f indicative of the distance to the target locations 150a, 150b, 150c, 150d, 150e, 150f, respectively, is communicated to the device 102.

In block 216, the device 102 determines if either in-plane or out-of-plane blade deflection is present during operation of the bone saw 106. To do so, the values of the measured distances from each of the sensors 148a, 148b, 148c, 148d, 148e, 148f are compiled and compared to known and/or expected distance values. The device 102 may use an algorithm, such as a smoothing algorithm, to process the values output from the sensors 148a, 148b, 148c, 148d, 148e, 148f and thereafter compare them to known and/or expected values. In such a way, the presence of blade deflection can be determined by the device 102 if the measured distance from one or a combination of sensors 148 varies outside of an accepted range (e.g., +/- 2mm). Given the array pattern of the sensors 148 (and corresponding pattern of the target areas 150), the amount and type (in-plane, out-of-plane, or both) of blade deflection may be determined by the device 102. If the device 102 determines that either no blade deflection is present – or that it is present within an acceptable range – the method loops back to block 210 to continue operation of the bone saw 106. However, if the device 102 determines that blade deflection is present outside of an acceptable range, the method advances to block 218.

In block 218, the device 102 adjusts operation of the bone saw 106. Based the amount and type (in-plane, out-of-plane, or both) of blade deflection and on a given system design, the specific operational adjustment of the bone saw 106 may be varied. For example, based on the amount and type (e.g., in-plane versus out-of-plane or both) of blade deflection, the surgical planning and assistance device 102 may adjust the oscillation speed of the saw blade 144 by changing the speed of the saw’s motor. In many such cases, the saw’s motor is slowed to slow the speed of the saw blade 144. In some cases, such as when out-of-plan blade deflection is detected, the surgical planning and assistance device 102 may operate the robotic surgical device 104 to adjust the cut angle of the bone saw 106 to accommodate for the detected amount of blade deflection. In some cases, in response to detected blade deflection, the surgical planning and assistance device 102 may operate the robotic surgical device 104 to slow the forward advancement of the bone saw 106 by the surgeon by providing resistance via the device’s robotic arm thereby reducing bending (and hence deflection) of the blade. In certain cases, the device 102 may cease operation of the bone saw 106.

In block 220, the device 102 also generates a warning message to the surgeon and other surgical personnel. For example, the surgical planning and assistance device 102 may operate the display 130 to generate a visual and/or audible feedback message to the surgeon that informs the surgeon or other surgical personnel of the amount and type (e.g., in-plane versus out-of-plane or both) of blade deflection. The method 200 then loops back to block 210 to continue operation of the bone saw 106.

After controlling the robotic surgical device 104 and the bone saw 106 to complete all of the planned resections, the method 200 is completed. The surgeon may then continue the orthopaedic surgical procedure, for example by installing one or more trial components, one or more prosthetic components, or otherwise completing the orthopaedic surgical procedure.

While the disclosure has been illustrated and described in detail in the drawings and foregoing description, such an illustration and description is to be considered as exemplary and not restrictive in character, it being understood that only illustrative embodiments have been shown and described and that all changes and modifications that come within the spirit of the disclosure are desired to be protected.

For example, although the concepts of the present disclosure have been described in the context of a total knee arthroplasty (TKA) procedure, the concepts of the present disclosure may be utilized in the performance of other knee arthroplasty procedures such as a unicompartmental knee arthroplasty (UKA) procedure. Moreover, in addition to knee arthroplasty procedures, the concepts of the present disclosure may also be utilized in other orthopaedic procedures such as hip replacement procedures including, for example, total hip arthroplasty (THA) procedures and shoulder replacement procedures such as total shoulder arthroplasty (TSA) procedures.

There are a plurality of advantages of the present disclosure arising from the various features of the apparatus, system, and method described herein. It will be noted that alternative embodiments of the apparatus, system, and method of the present disclosure may not include all of the features described yet still benefit from at least some of the advantages of such features. Those of ordinary skill in the art may readily devise their own implementations of the apparatus, system, and method that incorporate one or more of the features of the present invention and fall within the spirit and scope of the present disclosure.

1. A method of operating a robotic-assisted orthopaedic surgical system, the method comprising:
controlling, by a computer system, a bone saw to oscillate a bone saw blade,
operating, by the computer system, a sensor assembly positioned in the bone saw to determine an amount of blade deflection in the bone saw blade, and
adjusting, by the computer system, operation of the bone saw based on the determined amount of blade deflection of the bone saw blade.

2. The method of claim 1, wherein adjusting operation of the bone saw comprises adjusting, by the computer system, speed of oscillation of the bone saw blade.

3. The method of claim 2, wherein adjusting speed of oscillation comprises slowing speed of oscillation of the bone saw blade.

4. The method of claim 1, wherein adjusting operation of the bone saw comprises adjusting, by the computer system, a cutting angle of the bone saw blade.

5. The method of claim 1, wherein operating the sensor assembly positioned in the bone saw comprises operating, by the computer system, the sensor assembly positioned in the bone saw to determine an amount of in-plane blade deflection of the bone saw blade.

6. The method of claim 1, wherein operating the sensor assembly positioned in the bone saw comprises operating, by the computer system, the sensor assembly positioned in the bone saw to determine an amount of out-of-plane blade deflection of the bone saw blade.

7. The method of claim 1, wherein controlling the bone saw comprises controlling, by a computer system, the bone saw to oscillate the bone saw blade during advancement of the bone saw blade into a bone of a patient.

8. The method of claim 1, wherein operating the sensor assembly positioned in the bone saw comprises operating, by the computer system, the sensor assembly positioned in the bone saw to determine an amount of blade deflection in a linear array pattern on the bone saw blade.

9. The method of claim 1, wherein operating the sensor assembly positioned in the bone saw comprises operating, by the computer system, the sensor assembly positioned in the bone saw to determine an amount of blade deflection in a grid array pattern on the bone saw blade.

10. The method of claim 1, further comprising operating, by the computer system, a monitor device of the orthopaedic surgical system to generate an audible and/or visual feedback message based on the determined amount of blade deflection of the bone saw blade.

11. An orthopaedic surgical system, comprising:
a robotic-assisted bone saw, the bone saw having a sensor assembly housed therein, and
a computer system electrically coupled to the bone saw, the computer system being configured to:
control the bone saw to oscillate a bone saw blade,
operate the sensor assembly to determine an amount of blade deflection in the bone saw blade, and
adjust operation of the bone saw based on the determined amount of blade deflection of the bone saw blade.

12. The orthopaedic surgical system of claim 11, wherein the computer system is further configured to adjust speed of oscillation of the bone saw blade based on the determined amount of blade deflection of the bone saw blade.

13. The orthopaedic surgical system of claim 11, wherein the computer system is further configured to slow speed of oscillation of the bone saw blade based on the determined amount of blade deflection of the bone saw blade.

14. The orthopaedic surgical system of claim 11, wherein the computer system is further configured to adjust a cutting angle of the bone saw blade based on the determined amount of blade deflection of the bone saw blade.

15. The orthopaedic surgical system of claim 11, wherein the computer system is further configured to operate the sensor assembly positioned in the bone saw to determine an amount of in-plane blade deflection of the bone saw blade.

16. The orthopaedic surgical system of claim 11, wherein the computer system is further configured to operate the sensor assembly positioned in the bone saw to determine an amount of out-of-plane blade deflection of the bone saw blade.

17. The orthopaedic surgical system of claim 11, wherein the computer system is further configured to operate the bone saw to oscillate the bone saw blade during advancement of the bone saw blade into a bone of a patient.

18. The orthopaedic surgical system of claim 11, wherein the computer system is further configured to operate the sensor assembly positioned in the bone saw to determine an amount of blade deflection in a linear array pattern on the bone saw blade.

19. The orthopaedic surgical system of claim 11, wherein the computer system is further configured to operate the sensor assembly positioned in the bone saw to determine an amount of blade deflection in a grid array pattern on the bone saw blade.

20. The orthopaedic surgical system of claim 11, further comprising a monitor device, wherein the computer system is further configured to operate the monitor device to generate an audible and/or visual feedback message based on the determined amount of blade deflection of the bone saw blade.
